(19) United States
(12) Patent Application Publication
(10) Pub. No.: US 2023/0408473 A1
(43) Pub. Date: Dec. 21, 2023

(54) DETERMINATION METHOD AND FUEL CELL SYSTEM FOR DETECTING INFERIOR GAS

(71) Applicant: Robert Bosch GmbH, Stuttgart (DE)

(72) Inventors: Timo Bosch, Renningen (DE); Tobias Falkenau, Esslingen (DE)

(21) Appl. No.: 18/248,507

(22) PCT Filed: Sep. 29, 2021
(86) PCT No.: PCT/EP2021/076813

§ 371 Date: Apr. 10, 2023

(30) Foreign Application Priority Data
Oct. 14, 2020 (DE) 10 2020 212 963.0

(51) Int. Cl. G01N 33/00

(52) U.S. Cl. CPC G01N 33/0036

(57) ABSTRACT

The presented invention relates to a determination method (100) for determining an inferior gas component in a fuel for operating a fuel cell system (200). The determination method (100) comprises a control step (101) for operating the fuel cell system (200) in a determination mode at a constant operating point for a predefined period, a determination step (103) for determining a purge mass flow that is set during the determination mode, an ascertainment step (105) for ascertaining an inferior gas concentration in the fuel on the basis of the determined purge mass flow, and an output step (107) for outputting the ascertained inferior gas concentration on a display unit (209).

The presented invention also relates to a fuel cell system (200).

### BACKGROUND OF THE INVENTION

Polymer electrolyte membrane (PEM) fuel cell systems convert hydrogen by means of oxygen into electrical energy, generating waste heat and water.

A PEM fuel cell consists of an anode supplied with hydrogen, a cathode supplied with air, and a polymer electrolyte membrane placed therebetween. A plurality of individual fuel cells are stacked into a fuel cell stack in order to maximize a voltage to be generated. Within a fuel cell stack, there are supply channels that supply hydrogen and air to individual fuel cells as well as remove depleted, i.e., oxygen-poor, and humid air together with depleted, i.e., hydrogen-poor, anode exhaust gas.

A systemic approach for supplying hydrogen to a PEM anode has been established, in which still hydrogen-rich anode exhaust gas is again supplied to an anode entry by gas conveying units together with fresh hydrogen. This process is called recirculation. As gas conveying units, so-called “jet pumps” or hybrid solutions consisting of jet pumps and hydrogen fans are used.

Furthermore, it is known that nitrogen passes from the cathode side to the anode side by diffusion processes. Nitrogen serves as an inert gas for the electrochemical reaction taking place in the fuel cell. As an inert gas, nitrogen reduces a cell voltage of a fuel cell and, when present in a high concentration, can damage a fuel cell if it is no longer sufficiently supplied with hydrogen.

In the operation of a fuel cell system, operational situations regularly occur in which gas located in a recirculation space is discharged and displaced by fresh hydrogen in order to lower the nitrogen concentration in the recirculation space. This process is called purging.

Too frequent purging, while keeping nitrogen concentration low, reduces at the same time a system efficiency because fuel is wasted.

A source of, for example nitrogen, is a portion of inferior gas contained in the fuel. Because a pressure in a fuel cell system must be kept constant by introducing fuel, new inferior gas is also repeatedly conveyed into the fuel cell system, in particular into an anode space of the fuel cell system, during purging.

In addition to nitrogen, other inferior gases, such as argon, can be contained in the fuel.

It is known to mix purge gas into a cathode exhaust gas and to discharge it from a fuel cell system in a non-critical composition as exhaust gas. A hydrogen concentration in the discharged exhaust gas is measured by means of a hydrogen sensor.

### SUMMARY OF THE INVENTION

In the context of the presented invention, a determination method and a fuel cell system having the features of the respective independent claims are presented. Further features and details of the invention arise from the respective subclaims, the description, and the drawings. Of course, features and details described in connection with the determination method according to the invention also apply in connection with the fuel cell system according to the invention, and respectively vice versa, so that with respect to the disclosure, mutual reference to the individual aspects of the invention is or can always be made.

The presented invention serves to determine an inferior gas concentration in a fuel for operating a fuel cell system. In particular, the presented invention serves to display to a user of a fuel cell system an inferior gas concentration in a fuel that has been filled.

Thus, in a first aspect of the presented invention, a determination method for determining an inferior gas component in a fuel supplied to a fuel cell is presented. The determination method comprises a control step for operating the fuel cell system in a determination mode at a constant operating point for a predefined period, a determination step for determining a purge mass flow that is set during the determination mode, an ascertainment step for ascertaining an inferior gas concentration in the fuel on the basis of the determined purge mass flow, and an output step for outputting the ascertained inferior gas concentration on a display unit, and/or a setting step for setting the fuel cell system on the basis of the ascertained inferior gas concentration.

In the context of the presented invention, an inferior gas is understood as a non-hydrogen content in a fuel, i.e., a quantity of gases supplied to a fuel cell. In particular, the term inferior gas includes a concentration of nitrogen and/or argon and/or carbon dioxide and/or carbon monoxide.

The presented determination method is based on a determination mode of a fuel cell system in which the fuel cell system is operated at a constant operating point at which for example, a constant electric current is generated in a fuel cell stack of the fuel cell system and a constant amount of exhaust gas is discharged from the fuel cell system. Accordingly, in the determination mode of the fuel cell system, there are constant operating conditions at the constant operating point, so that parameters, i.e., measured variables that change during the determination mode, such as a hydrogen concentration in the exhaust gas or an activation frequency of the purge valve, can be attributed to a change in the fuel.

In the determination mode of the presented determination method, it is possible to discern from a hydrogen concentration in the exhaust gas of a fuel cell system whether there is a stationary or constant inferior gas concentration in an anode path of the fuel cell system, because, in a cyclic operation of the purge valve or a constant activity of the purge valve, hydrogen concentrations of a constant size are set in the exhaust gas of the fuel cell system when there is a constant inferior gas concentration in the anode path of the fuel cell system.

When a purge valve is set or activated as a function of a hydrogen concentration in the exhaust gas of a fuel cell system, an inferior gas concentration in the fuel can be inferred on the basis of a purge mass flow that is influenced by an activity of a purge valve of a respective fuel cell system.

As soon as the inferior gas concentration has been determined, it can be output on an outputting unit, such as a display, in particular a display in a vehicle, a display of a mobile computing unit, or a display of a respective fuel cell system. Alternatively or additionally, on the basis of the determined inferior gas concentration, the respective fuel cell system can be set by determining, for example, a concentration of respective operating media supplied to the fuel cell system using the inferior gas concentration. Accordingly, the inferior gas concentration can be transferred as a transfer value to a target function.

In particular, it is provided that the presented determination method is performed after a fueling operation in order to fill a tank of a fuel cell system with fuel so as to sense an entry of inferior gas through the fueling operation.

It can be provided that the purge mass flow is determined on the basis of a number of performed purge cycles.

It is provided in particular that a purge mass flow or an activity of a purge valve is controlled or activated as a function of a hydrogen concentration ascertained in the exhaust gas of a respective fuel cell system. It has been shown that too much activity of the purge valve or too high a purge frequency results in a continuously increasing concentration of hydrogen in the exhaust gas of a fuel cell system, and too little activity or purge frequency of the purge valve results in a continuously decreasing concentration of hydrogen in the exhaust gas of the fuel cell system in the determination mode. Accordingly, it can be provided that the activity of the purge valve is used as a control variable in order to constantly regulate a concentration of hydrogen in an exhaust gas of the fuel cell system so that an inferior gas concentration in the supplied fuel can be inferred.

However, when a purge valve is controlled or activated as a function of a hydrogen concentration measured in the exhaust gas of a fuel cell system such that a constant hydrogen concentration in the exhaust gas of the fuel cell system is set, the hydrogen concentration in the exhaust gas and the activity of the purge valve are correlated with one another. Accordingly, in the embodiment of the presented invention, it can be provided that an inferior gas concentration, in particular of nitrogen, in a fuel supplied to the fuel cell system can be inferred from a purge mass flow that is influenced by an activity of the purge valve.

It can further be provided that the purge mass flow is ascertained by means of a mass flow sensor.

Because an activity or frequency of the activity of the purge valve correlates with a purge mass flow, i.e., a mass flow of media discharged through the purge valve, the purge mass flow can be inferred from the activity of the purge valve. Alternatively, the purge mass flow can be measured directly by means of a sensor.

As soon as a purge mass flow is known, i.e., has been determined for example on the basis of a purge frequency of purge operations performed by a purge valve in the determination mode, at a constant exhaust gas concentration, i.e., a consistent hydrogen concentration in the exhaust gas of a respective fuel cell system over a predefined minimum period of time, an inferior gas concentration in a fuel used during the determination mode can be ascertained on the basis of the purge mass flow. For this purpose, for example, a previously experimentally determined allocation scheme can be used, which allocates a value of an inferior gas concentration to a respective value of a purge mass flow.

For example, an allocation scheme can comprise a mathematical formula that maps a linear or non-linear relationship in order to allocate a value of an inferior gas concentration to a value of a purge mass flow ascertained in the determination mode of a respective fuel cell system. The mathematical formula can comprise a mathematical model, for example, which adapts a relationship between an ascertained value of a purge mass flow and a value of an inferior gas concentration as a function of a parameter, for example a system temperature and/or an ambient temperature. In particular, the mathematical model can assume that, for example, knowing a stack flow, a moisture assumed to be 100% relative humidity at a known temperature, and a predefined nitrogen crossover rate, a purge mass flow for maintaining a constant nitrogen concentration in the anode path only depends on an impurity concentration in the fuel.

It can further be provided that, during the determination mode, a purge frequency of a purge valve of the fuel cell system is varied until a hydrogen concentration in an exhaust gas generated by the fuel cell system is constant, and then, when the hydrogen concentration in the exhaust gas is constant, the purge mass flow is subsequently determined.

A purge frequency, i.e., a frequency at which a purge valve is activated, can be increased or decreased, for example by means of an automatic control loop, until a hydrogen concentration in the exhaust gas of a respective fuel cell system is constant.

It can be provided that, in the determination mode, the activation frequency of the purge valve is reduced with a decreasing concentration of hydrogen in the exhaust gas and increased with an increasing concentration of hydrogen in the exhaust gas.

At a constant operating point in the determination mode, the purge frequency can, for example, be decreased when the hydrogen concentration in the exhaust gas decreases between two successive purge events and increased when the hydrogen concentration in the exhaust gas increases between two successive purge events. As soon as the hydrogen concentration in the exhaust gas is constant or a constant purge frequency is set, i.e., the purge frequency does not change over e.g. at least two purge operations, the inferior gas concentration can be ascertained on the basis of the constant purge frequency.

In a second aspect, the presented invention relates to the use of a possible configuration of the presented determination method for displaying a quality of a fuel on a display unit.

An inferior gas concentration ascertained by the presented determination method can be used in order to determine a quality of a fuel and output it on a display unit. For this purpose, a characteristic value of a quality can be allocated to a respective value of an inferior gas concentration, for example by means of an allocation scheme. In particular, the characteristic value can correspond to the ascertained value of the inferior gas concentration. The characteristic value can be shown in color according to a predefined scheme or scale in order to be able to assess the quality of the fuel in relation to a standard.

In a third aspect, the present invention relates to the use of a possible configuration of the present determination method for ascertaining an inferior gas concentration in a tank system for providing fuel for a fuel cell system.

By a sampling of a fuel provided by a tank system, a purity of the tank system of inferior gas can be assessed. Accordingly, a tank system can be inspected for leakage or residual gases in respective conduits of the tank system by means of a fuel cell system configured so as to perform the presented determination method, because these would be shown by an increased inferior gas concentration in a fuel. In particular, in a first step, the determination method can be performed by means of a fuel provided directly or by a tank system known as “inferior-gas-free,” and, in a second step, with an identical fuel provided by a tank system to be inspected, the determination method can be performed so that, if the inferior gas concentration deviates between the first step and the second step, it can be assumed that the deviation is caused by the tank system to be inspected.

In a fourth aspect, the presented invention relates to a fuel cell system with an inferior gas determination. The fuel cell system comprises a fuel cell stack, a hydrogen sensor for measuring a hydrogen concentration in an exhaust gas of the fuel cell system, a purge valve, and a control device, wherein the control device is configured so as to operate the fuel cell system in a determination mode at a constant operating point for a predefined period, determine a purge mass flow that is set by means of the purge valve during the determination mode, ascertain an inferior gas concentration in a fuel supplied to the fuel cell system on the basis of the determined purge mass flow, and output the ascertained inferior gas concentration on a display unit.

In a fifth aspect, the presented invention relates to using a possible configuration of the presented determination method for setting a fuel cell system to an optimal operating point.

In particular, a stationary operating point used in a fuel specifically for detecting a gas quality or an inferior gas concentration can be defined, which is set for example after the fueling operation. Once a tank of a fuel cell system has been filled, the tank is generally homogeneously emptied, i.e., there is no in-tank demixing, and the inferior gas concentration remains constant over the removal process. Accordingly, an inferior gas concentration ascertained after a tank operation can be used in order to calibrate the fuel cell system, in particular an anode subsystem, in order to set respective operating parameters of the fuel cell system.

It can be provided that, in a use of tank systems in which there can be a demixing of respective inferior gases and respective fuel, a correction term for correcting an ascertained inferior gas concentration according to the invention is used. For example, the correction term can mathematically map a demixing in a tank system as a function of a time elapsed since a tank operation.

The presented fuel cell system serves in particular to perform the presented determination method.

It can be provided that the control device is configured so as to transfer respectively ascertained inferior gas concentrations via an output interface to a central server in order to provide the respectively ascertained inferior gas concentrations for computing units connected to the central server.

### BRIEF DESCRIPTION OF THE DRAWINGS

Further advantages, features, and details of the invention arise from the following description, in which various exemplary embodiments of the invention are described in detail with reference to the drawings. The features mentioned in the claims and in the description can be essential to the invention individually or in any combination.

The figures show:

FIG. 1 a possible configuration of the presented determination method,

FIG. 2 a possible configuration of the presented fuel cell system.

### DETAILED DESCRIPTION

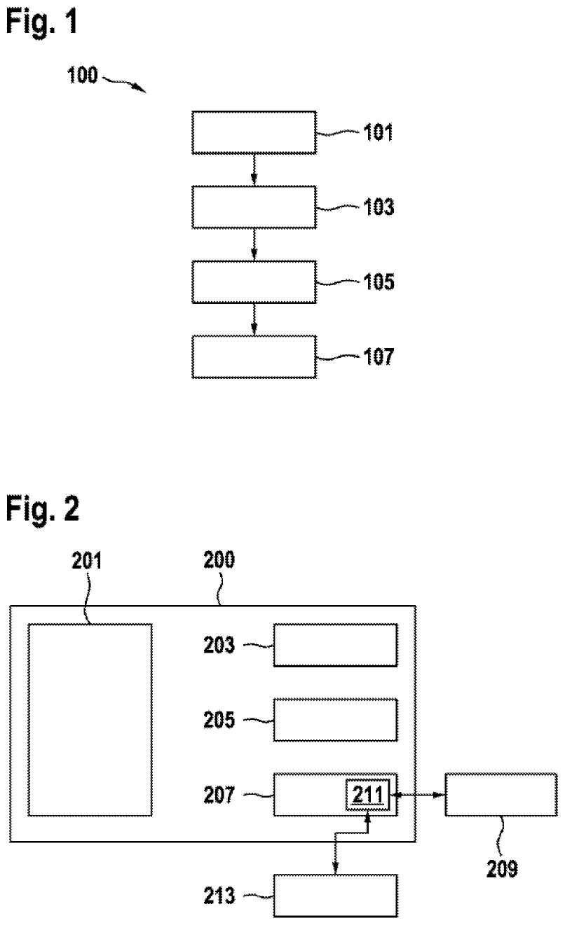

In FIG. 1, a determination method 100 is shown. The determination method comprises a control step 101 for operating a fuel cell system in a determination mode at a constant operating point for a predefined period. The fuel cell system is operated with a constant electric current in the fuel cell stack and with a constant quantity of exhaust gas air. Furthermore, a hydrogen concentration in the exhaust gas of the fuel cell system is determined continuously or at regular intervals.

Furthermore, the determination method 100 comprises a determination step 103 for determining a purge mass flow that is set during the determination mode. For example, a purge frequency at which a purge valve of the fuel cell system is activated is sensed. Alternatively, a mass flow sensor can be used in the exhaust gas system of the fuel cell system in order to measure the purge mass flow.

As soon as the fuel cell system is operated at a constant operating point in the determination mode, the purge frequency at which the purge valve is activated is changed as a function of a hydrogen concentration measured in the exhaust gas of the fuel cell system. For this purpose, the purge frequency can be decreased as the hydrogen concentration decreases or the purge frequency can be increased as the hydrogen concentration increases.

As soon as a hydrogen concentration in the exhaust gas of the fuel cell system is constant during the determination mode, an ascertainment step 105 is performed in which an inferior gas concentration in the fuel is determined on the basis of a purge mass flow that is set by the purge valve. For example, a hydrogen concentration can be constant if it does not change between two purging operations or only changes by less than a predefined amount.

In order to ascertain the inferior gas concentration, a value of an inferior gas concentration can be allocated to an ascertained value of the purge mass flow, e.g. by means of a predefined allocation scheme. The allocation scheme can be ascertained in advance, for example by means of test setups and/or can comprise a mathematical formula that mathematically models or maps a relationship between the purge mass flow and the inferior gas concentration, in particular using the parameters of electrical current in the fuel cell stack, nitrogen transfer from the anode to the cathode, and a moisture in the anode path.

An inferior gas concentration ascertained by the determination step is output on a display unit in an outputting step 107. Alternatively or additionally, the ascertained inferior gas concentration can be used in order to calibrate the fuel cell system, for example, so as to adjust respective operating parameters of the fuel cell system to the ascertained inferior gas concentration.

FIG. 2 shows a fuel cell system 200. The fuel cell system 200 comprises a fuel cell stack 201, a hydrogen sensor 203 for measuring a concentration of hydrogen in an exhaust gas of the fuel cell system 200, a purge valve 205, and a control device 207.

The control device 207, which can be for example a control device of the fuel cell system 200 or any other programmable computing unit, is configured so as to operate the fuel cell system 200 in a determination mode at a constant operating point for a predefined period, determine a purge mass flow that is set during the determination mode using the purge valve 205, ascertain an inferior gas concentration in a fuel supplied to the fuel cell system 200 on the basis of the determined purge mass flow, and output the ascertained inferior gas concentration on a display unit 209.

In order to output the ascertained inferior gas concentration, the control device 209, which can be for example a processor, a computer, a controller, an ASIC, or any other programmable element, can be communicatively connected with the display unit 209 via an output interface 211. In the present case, the display unit 209 is a smartphone of a user of the fuel cell system 200.

For example, the user can be shown on the display unit 209 a path of the inferior gas concentration over time and/or over various tank operations or tank systems such that the user can discern tank systems with particularly low or particularly high concentrations of inferior gas.

Via the output interface 209, respectively ascertained inferior gas concentrations can be transmitted to an optional central server 213, such as a cloud server. The inferior gas concentrations stored on the central server 213 can be transferred to computing units connected to the central server 213, such as smartphones, in order to inform, for example, further users about the ascertained inferior gas concentrations of the respective tank systems.

1. A determination method (100) for determining an inferior gas component in a fuel for operating a fuel cell system (200), the method (100) comprising:
operating (101) the fuel cell system (200), via a control device (207), in a determination mode at a constant operating point for a predefined period,
determining (103), via the control device (207), a purge mass flow that is set during the determination mode,
ascertaining (105), via the control device (207) an inferior gas concentration in the fuel on the basis of the determined purge mass flow,
outputting (107) the ascertained inferior gas concentration on a display unit (209) and/or setting the fuel cell system, via the control device 207) on the basis of the ascertained inferior gas concentration.

2. The determination method (100) according to claim 1,
wherein
the purge mass flow is determined on the basis of a number of performed purge cycles.

3. The determination method (100) according to claim 1,
wherein
the purge mass flow is determined by means of a mass flow sensor.

4. The determination method (100) according to claim 1,
wherein
the inferior gas concentration is determined by means of an allocation scheme that mathematically maps an association between a purge mass flow and an inferior gas concentration for the fuel cell system (200).

5. The determination method (100) according to claim 1,
wherein
during the determination mode, a purge frequency of a purge valve (205) of the fuel cell system (200) is varied until a hydrogen concentration in an exhaust gas generated by the fuel cell system (200) is constant, and
wherein, when the hydrogen concentration in the exhaust gas is constant, the purge mass flow is subsequently determined.

6. The determination method (100) according to claim 5,
wherein
in the determination mode, the activation frequency of the purge valve (205) is reduced with a decreasing concentration of hydrogen in the exhaust gas and increased with an increasing concentration of hydrogen in the exhaust gas.

7. A use of the determination method (100) according to claim 1 for indicating a quality of a fuel on a display unit (209).

8. The use of the determination method (100) according to claim 1 for determining an inferior gas concentration in a tank system for providing fuel to a fuel cell system.

9. The use of the determination method (100) according to claim 1 for setting a fuel cell system to an optimal operating point.

10. A fuel cell system (200) with an inferior gas determination,
wherein the fuel cell system (200) comprises:
a fuel cell stack (201),
a hydrogen sensor (203) for measuring a hydrogen concentration in an exhaust gas of the fuel cell system (200),
a purge valve (205),
a control device (207),
wherein the control device (207) is configured so as to:
operate the fuel cell system (200) in a determination mode at a constant operating point for a predefined period,
determine a purge mass flow that is set by means of the purge valve (205) during the determination mode,
ascertain an inferior gas concentration in a fuel supplied to the fuel cell system (200) on the basis of the determined purge mass flow, and
output the ascertained inferior gas concentration on a display unit (209).

11. The fuel cell system (200) according to claim 10,
wherein the control device (207) is configured so as to transfer respectively ascertained inferior gas concentrations via an output interface (211) to a central server (213) in order to provide the respectively ascertained inferior gas concentrations for computing units connected to the central server (213).
